(19) United States
(12) Patent Application Publication
(10) Pub. No.: US 2020/0217852 A1
(43) Pub. Date: Jul. 9, 2020

(54) Quantitation of Free and Total N-Acetylcysteine Amide and Its Metabolite N-Acetylcysteine in Human Plasma Using Derivatization and Electrospray LC-MS/MS

(71) Applicant: NACUITY PHARMACEUTICALS, INC., Fort Worth, TX (US)

(72) Inventors: G. Michael Wall, Fort Worth, TX (US); Brad King, Indianapolis, IN (US)

(21) Appl. No.: 16/736,681

(22) Filed: Jan. 7, 2020

Related U.S. Application Data
(60) Provisional application No. 62/790,344, filed on Jan. 9, 2019.

(51) Int. Cl. G01N 33/68

(52) U.S. Cl. CPC G01N 33/6815; G01N 2800/50; G01N 2800/164; G01N 33/6848

(57) ABSTRACT

The present invention includes a method of detecting free and total NAC, NACA, or both in a biological sample and/or the effectiveness of a treatment with NAC, NACA, or diNACA comprising: adding 2-chloro-1-methylpyridinium iodide (CMPI) to a biological sample suspected of having NAC or NACA to convert free thiols into stable thioethers; precipitating the protein in the sample; extracting the stable thioethers and separating into a first and a second extract; detecting the thioether derivatives from the first extract with LC-MS/MS; reducing from the second extract free thiols by adding tris(2-carboxyethyl)phosphine (TCEP) followed by converting to stable thioethers with CMPI; detecting the disulfides reduced to free thioether derivatives from the second extract with LC-MS/MS; and calculating from the LC-MS/MS and TCEP of the first and second extracts a free and a total sample NAC or NACA.

### CROSS-REFERENCE TO RELATED APPLICATIONS

This application claims priority to U.S. Provisional Application Ser. No. 62/790,344, filed Jan. 9, 2019, the entire contents of which are incorporated herein by reference.

### TECHNICAL FIELD OF THE INVENTION

The present invention relates in general to the field of detection of active agents from free and total N-acetylcysteine amide and its metabolite, N-acetylcysteine, in human plasma using derivatization and electrospray LC-MS/MS, and the use of the same to show the effectiveness of a treatment with N-acetylcysteine, N-acetylcysteine amide, di-N-acetylcysteine amide, and derivatives thereof.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with detection of N-acetyl cysteine.

N-acetyl-L-cysteine (NAC) is a well-known, endogenous antioxidant moiety that can facilitate glutathione biosynthesis and replenish glutathione within cells that are under oxidative stress. NAC is FDA-approved for the treatment of acetaminophen overdose (ACETADOTE, CETYLEV) and as a mucolytic (MUCOMYST (now generic)) [1]. N-acetylcysteine amide (NACA), synthesized by Martin et al. [2] for potential use as a mucolytic agent but not yet approved by any regulatory agency, is the amide form of NAC (FIG. 1). NACA is more lipophilic and more easily permeates cell membranes than NAC. In studies with mice it has shown great potential for crossing the blood-brain and retinal barriers. A recent keyword search for “N-acetylcysteine” in PubMed revealed 17,376 hits, including a plethora of uses associated with its antioxidant activity. While there are far fewer studies published on NACA, a recent review by Sunitha et al. (2013) [3] described numerous effects of NACA in cells or animal models including antioxidant, anti-apoptotic, anti-inflammatory and neuroprotective effects. These effects appear to be due to the ability of NACA to reduce reactive oxygen species, chelate heavy metals and prevent formation of pro-inflammatory cytokines. NACA has also shown protection against oxidative stress-mediated ophthalmic effects [4,5] and is being developed as a potential treatment for retinitis pigmentosa.

A commonly used index of tissue oxidative stress is the concentration ratio of glutathione (GSH) to its disulfide (GSSG); accurate measurement of this ratio requires that both thiol and disulfide be measured without disturbing the redox balance at time of collection. At physiological pH, thiols are rapidly oxidized in vitro after blood collection and the ratio can dramatically shift. Extensive reviews [6-8] have summarized the dozens of publications for the measurement of endogenous thiols and disulfides. Inhibiting oxidation by chilling the sample, working rapidly to collect and freeze the sample, and using various chemical measures (lowering the pH, adding chelating agents, or masking the reactive thiol to form a more stable species) have all been employed [9-13]. Thiols will readily react with N-ethylmaleimide, iodoacetamide, monobromobimane, 2-halopyridinium salts, and other agents to form stable derivatives suitable for liquid chromatography with ultraviolet absorbance, fluorescence or MS/MS detection. The reaction conditions must be optimized to mask all thiols quickly, or else some thiols may be partially converted to disulfides and cause inaccurate ratio measurements. Ideally, the reaction should be completed in seconds. Customized maleimides [7] such as charged derivatives for electrospray MS detection have been advanced to not only provide stability but also to improve sensitivity. Reactive 2-halopyridinium salts have been similarly optimized in methods employing HPLC with UV detection [8, 13] to create stable thioether derivatives with favorable bathochromic shifts. Disulfides are typically measured directly or after reduction by borohydride, dithiothreitol (DTT), or trialkylphosphines such as tris(2-carboxyethyl)phosphine (TCEP).

While these chemistries were largely developed to measure endogenous thiols such as cysteine, homocysteine, and glutathione, fewer publications have reported on the measurement of NAC and NACA. Ercal et al. developed a method [14] in plasma for NAC using N-(1-pyrenyl)maleimide (NPM) to form a stable adduct; any disulfides were reduced in a second sample with DTT and then reacted with NPM to measure total thiol, with HPLC and fluorescence detection. Wu et al. later used the same NPM chemistry and HPLC with fluorescence detection to measure reduced NAC and NACA in rat plasma and tissue; glutathione, cysteine, and homocysteine were also monitored [15]. The authors demonstrated that plasma concentrations of NAC, NACA, GSH, and cysteine in plasma were sharply increased 30 minutes after oral administration of NACA 500 mg/kg, and tissue concentrations were elevated to a lesser extent in kidney, lung, brain and liver, all compared to the controls. Nozal et al. separated native GSH, cysteine, and NAC in rabbit eye tissues on conventional reverse phase columns and then used a post-column reactor with 5,5′-dithiobis(2-nitrobenzoic acid) to form fluorescent derivatives [16]. Celma et al. [17] measured total NAC in human plasma by DTT reduction, liquid-liquid extraction, and LC-MS/MS analysis of the thiol without derivatization. Katz et al. [18] and Reyes at al. [19] successfully used TCEP to reduce disulfides and N-(9-acridinyl) maleimide to form stable thiol adducts of NAC, cysteine, and GSH measured by LCMS in human cerebrospinal fluid.

However, a need remains for accurate determination of both serum and plasma levels of NAC, NACA or di-NACA, in particular total levels of NAC and/or NACA.

### SUMMARY OF THE INVENTION

In one embodiment, the present invention includes a method of detecting free and total NAC, NACA, or both in a biological sample comprising: adding 2-chloro-1-methylpyridinium iodide (CMPI) to a biological sample suspected of having NAC or NACA to convert free thiols into stable thioethers; precipitating the protein in the sample; extracting the stable thioethers and separating into a first and a second extract; detecting the thioether derivatives from the first extract with LC-MS/MS; reducing from the second extract free thiols by adding tris(2-carboxyethyl)phosphine (TCEP) followed by converting to stable thioethers with CMPI; detecting the disulfides reduced to free thioether derivatives from the second extract with LC-MS/MS; and calculating from the LC-MS/MS and TCEP of the first and second extracts a free and a total sample NAC or NACA. In one aspect, the biological sample is a plasma, serum, vitreous humor, tear, sputum, urine, or fecal sample. In another aspect, the LC-MS/MS is Liquid Chromatography/Triple Quadrupole Mass Spectroscopy. In another aspect, the results provide total assay measures a sum of free plus oxidized NAC or NACA. In another aspect, the method further comprises the step of optimizing the thiol and disulfide measurements by acidifying the sample prior to adding CPMI. In another aspect, the method further comprises the step of performing the ionization at a spray voltage of 5000 V, vaporizer temperature of 400° C., and capillary temperature or 250° C.

In another embodiment, the present invention includes a method comprising: measuring by Liquid Chromatography/Triple Quadrupole Mass Spectroscopy (LC-MS/MS) the levels of total NAC, NACA, or di-NACA in a biological sample obtained from a human subject having retinitis pigmentosa, age-related macular degeneration, diabetic retinopathy, myopia, high myopia, Fuchs' dystrophy, diabetic macular edema (DME), geographic atrophy, Stargardt's disease, cataracts, or retinal vein occlusion (RVO), by: adding 2-chloro-1-methylpyridinium iodide (CMPI) to the biological sample suspected of having NAC or NACA to convert free thiols into stable thioethers; precipitating the protein in the sample; extracting the stable thioethers and separating into a first and a second extract; detecting the thioether derivatives from the first extract with LC-MS/MS; reducing from the second extract free thiols by adding tris(2-carboxyethyl)phosphine (TCEP) followed by converting to stable thioethers with CMPI; detecting the disulfides reduced to free thioether derivatives from the second extract with LC-MS/MS; and calculating from the LC-MS/MS and TCEP of the first and second extracts a free and a total sample NAC, NACA. or di-NACA. In one aspect, the biological sample comprises a plasma, serum, vitreous humor, tear, sputum, urine, or fecal sample. In another aspect, the human subject is determined to be at risk of developing retinitis pigmentosa or a disorder associated with the eye. In another aspect, the human subject is determined to be at risk of developing retinitis pigmentosa or a disorder associated with the eye. In another aspect, the human subject is determined to be at risk of developing complications from retinitis pigmentosa or a disorder associated with the eye. In another aspect, the method further comprises calculating the risk or rate of the human subject developing retinitis pigmentosa or a disorder associated with the eye, wherein the risk or rate is calculated based on probability and odds ratios of developing biopsy documented retinitis pigmentosa. In another aspect, the method further comprises providing recommended treatment options for the human subject based on the calculated risk or rate of developing retinitis pigmentosa or a disorder associated with the eye. In another aspect, the method further comprises compiling the calculations of the risk or rate of developing retinitis pigmentosa or a disorder associated with the eye in the human subject into a report. In another aspect, the report is transmitted to a third party or to the human subject. In another aspect, the transmitting of the report is done over a network. In another aspect, the report comprises a risk profile. In another aspect, the report is transmitted to a third party and to the subject. In another aspect, the human subject has not been diagnosed with retinitis pigmentosa or a disorder associated with the eye. In another aspect, a third party obtains the plasma sample from the subject. In another aspect, the human subject is undergoing treatment with at least one of NAC, NACA, or diNACA.

### BRIEF DESCRIPTION OF THE DRAWINGS

For a more complete understanding of the features and advantages of the present invention, reference is now made to the detailed description of the invention along with the accompanying figures and in which:

FIG. 1 shows the structures and reaction of NAC and NACA with CMPI to form stable derivatives.

FIG. 2 shows the reduction of NAC or NACA disulfide with TCEP to yield free thiols.

FIG. 3 shows the fragmentation scheme for NAC thioether derivative.

FIGS. 4A to 4C show the NAC and NAC-d3 derivative chromatograms from plasma extracts of (FIG. 4A) blank, (FIG. 4B) 50 ng/mL lower limit calibration standard, and (FIG. 4C) a high QC sample (37.5 μg/mL). Analyte is presented on left panel and internal standard on right panel. The insignificant noise peak at 2.18 minutes (47 area counts) was auto marked because it was largest peak found in the retention time window.

FIGS. 5A and 5B show NACA and NACA-d3 derivative chromatograms from plasma extracts of (FIG. 5A) blank with internal standard, (FIG. 5B) a low validation/QC sample (150 ng/mL). Analyte is presented on left panel and internal standard on right panel.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The present inventors described herein the development and validation of an assay for free and total NAC and NACA in human plasma. The novel method can be used in conjunction with the treatment of a subject with at least one of NAC, NACA, or diNACA, for example, to determine the effectiveness of a treatment with NAC, NACA, or diNACA of, e.g., retinitis pigmentosa, age-related macular degeneration, diabetic retinopathy, myopia, high myopia, Fuchs' dystrophy, diabetic macular edema (DME), geographic atrophy, Stargardt's disease, cataracts, or retinal vein occlusion (RVO). The method is a novel derivatization, protein precipitation extraction, and LC-MS/MS instrumental analysis. At the time of sample collection, plasma will contain a mixture of NAC and NACA in both their reduced (RSH) and oxidized forms (RSSR, RSSR′). The oxidized forms may consist of symmetrical (e.g., NAC-NAC) and mixed disulfides (e.g., NAC-NACA, NACA-glutathione, NAC-protein conjugates). Since reference standards are not available for most of these disulfides, the assay is designed to measure total NAC and NACA rather than individual disulfides.

Samples can be assayed for free, total, or both free plus total NAC and NACA. An LC-MS/MS detection scheme was used to adapt the method to the measurement of NAC and NACA in ocular tissue and humor.

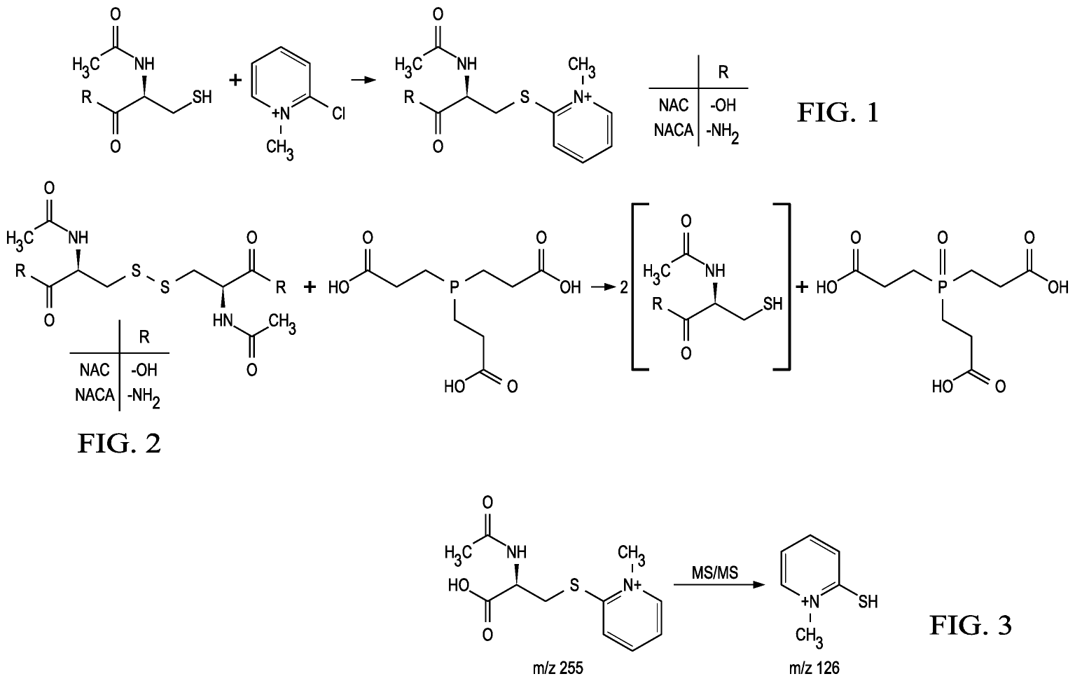

In the method of the present invention, free thiols are converted to stable thioethers at the time of blood collection using 2-chloro-1-methylpyridinium iodide (CMPI) as a derivatizing reagent (FIG. 1); this step prevents oxidative losses of the thiols immediately after collection. From the harvested plasma, the thioether derivatives are measured by LC-MS/MS after protein precipitation extraction. From another portion of the same extraction, disulfides are reduced to free thiols by TCEP (FIG. 2) and reacted with CMPI after which sample protein is precipitated and the supernatant injected separately. The total assay measures the sum of free plus oxidized NAC or NACA. The same thioether derivatives are detected by LC-MS/MS for both the free and total assays.

Reference Standards. NAC, its d3-labelled isotope (NAC-d3), and N, N′-diacetyl-L-cystine were purchased from Toronto Research Chemicals, North York, Ontario, Canada (A172091, A172092, and D312500, respectively). NACA, NACA disulfide (di-NACA) and NACA-d3-labelled isotope were provided by Nacuity Pharmaceuticals Inc., Ft. Worth, Tex. All were stored at 2-8° C.

Chemicals and other critical materials. 2-Chloro-1-methylpyridinium iodide (CMPI) and tris(2-carboxyethyl)phosphine (TCEP) were sourced from Sigma-Aldrich (198005 and C4706, respectively). Acetonitrile, methanol, ammonium bicarbonate, formic acid, ammonium hydroxide solution, and acetic acid were supplied by Fisher Scientific or Thermo Fisher (all Optima LC/MS grade).

Equipment. Extractions were performed using a Tomtec Quadra 96 system (Tomtec, Hamden, Conn.). Empty polypropylene 1.2 mL 96-well plates and cap mats were provided by Thermo. Samples were analyzed on a Waters Acquity liquid chromatograph interfaced with a Thermo Scientific TSQ Vantage triple quadrupole mass spectrometer with ionization in positive ion mode. A Waters ethylene-bridged hybrid stationary phase for hydrophilic-interaction chromatography (BEH HILIC, 2.1×100 mm, 1.7 μm) was utilized.

Matrix. Human K2EDTA plasma was obtained from Bioreclamation (New York). For some QC sample pools, the plasma was pre-fortified with CMPI (1.5 M aqueous CMPI:plasma, 1:99 by volume). This preparation is only good for a single day and should be prepared fresh on the day of use.

Calibration. Separate stock solutions of NAC and NACA (5.00 mg/mL) were prepared in 0.5 mg/mL disodium EDTA (pH 7.0) in amber glass vials and maintained at 2-8° C. for up to 22 days. The stock solutions were diluted with 0.5 mg/mL disodium EDTA:formic acid (100:0.1) to make combined NAC/NACA spiking solutions of 1000, 800, 400, 100, 50.0, 10.0, 2.00, and 1.00 μg/mL. Acidifying the diluent with formic acid stabilized the spiking solutions for one week.

Calibration standards (CS) were prepared by mixing CMPI-fortified control matrix (95 parts) with the respective spiking solution (5 parts) to make 50.0, 40.0, 20.0, 5.00, 2.50, 0.500, 0.100, and 0.050 μg/mL NAC/NACA pools which were stable for at least 80 days when stored at −20° C. and for 284 days when stored at −80° C. It was critical that CS pools be spiked quickly after CMPI fortification of the plasma, with the lowest concentration pools being spiked first, within 5-10 minutes of mixing plasma with CMPI.

Validation/quality control samples. To verify the accuracy and precision of the assay, two types of QC samples were prepared which differed only as to whether the matrix was fortified with CMPI. The fortified QC samples would be expected to be accurately measured for both free and total NAC and NACA. The unfortified QC samples would likely demonstrate negative bias in the free assay due to oxidation of the unprotected thiol but should be measured accurately in the total assay. Including both types of QC samples in each analytical run would prove that the derivatization steps were effective.

The quality control samples (QC) were prepared from separate 5.0 mg/mL NAC and NACA stock solutions which were diluted to prepare combined 750, 75.0, 3.00, and 1.00 μg/mL spiking solutions in 0.5 mg/mL disodium EDTA:formic acid (100:0.1). These spiking solutions were then diluted (95:5) with either fortified or unfortified matrix to create 37.5, 3.75, 0.150, and 0.050 μg/mL validation sample pools. Spiking the analytes in a fortified matrix was accomplished rapidly after CMPI fortification of the control matrix.

Internal standard solution. Separate 1.0 mg/mL stock solutions of NAC-d3 and NACA-d3 in 0.5 mg/mL disodium EDTA (pH 7.0) in amber glass vials were diluted in water to make a spiking solution containing both analytes (1.0 μg/mL), good for 60 days when stored at 2-8° C.

Blood collection. Whole blood was collected in K2EDTA blood collection tubes and immediately uncapped to allow 1.5 M aqueous CMPI solution (1% by nominal collection volume) to be added. The tubes were recapped, gently mixed by inversion, and allowed to react for 10 minutes at room temperature before the plasma was harvested. There was no distinction in the extent of hemolysis from added CMPI solution compared to adding water.

Extraction procedure. A 25 μL aliquot of calibration standard, QC sample, control, or unknown sample was mixed with 5.0 μL CMPI (60 mM) and 25.0 μL internal standard solution in a 1.2 mL microtiter plate (plate #1) and allowed to react at room temperature for approximately 10 minutes to allow derivatization of the internal standards. 50.0 μL of 100 mM ammonium bicarbonate was then added to each sample well and the plate was briefly vortexed and centrifuged.

A 50 μL aliquot from each well was transferred to a new plate (#2), to which 5.0 μL CMPI (60 mM) and 5.0 μL TCEP (60 mM) were immediately added. Plate #2 was vortexed and briefly centrifuged, then allowed to react at room temperature for 30 minutes to reduce any disulfides and convert the resulting thiols to protected thioethers.

Acetonitrile (500 μL) was then added to each well of plates #1 and #2 to precipitate proteins. The wells were covered with a capmat and shaken vigorously for 1 minute and then centrifuged for 2 minutes at 1500 ref. From both plates #1 and #2, the Tomtec handler transferred 50 μL of supernatant to new plates #3 and #4, respectively, already containing 300 μL water:acetonitrile (25:75). The plates were mixed by repetitive aspiration using the Tomtec and then sealed for injection. Plate #3 was injected to measure free NAC and NACA, whereas plate #4 was injected to assess total NAC and NACA concentrations.

Instrumental Analysis Procedures. Each extracted sample was injected (5.0 μL) onto a BEH HILIC column equilibrated at 35° C. Mobile phase A was 25 mM ammonium formate, pH 3.8, and mobile phase B was acetonitrile. The mobile phase was delivered at 0.50 mL/min and fixed at 25% A:75% B for the entire runtime. Retention times were approximately 1.1-1.2 minutes for CMPI-NACA and 1.9 min for CMPI-NAC.

For the CMPI derivative of NAC, the mass transitions (singly charged) were 255.1→126.2 (2-thio-N-methylpyridinium ion) and 258.1→126.2 for its internal standard (see FIG. 3 for fragmentation scheme). For the NACA derivative and its internal standard, the transitions were 254.1→126.2 and 257.1→126.2, respectively. Peak area ratios from the calibration standard responses were regressed using a (1/concentration2) linear fit for both NAC and NACA.

Ionization Optimization. Both the NAC-CMPI and NACA-CMPI derivatives were measured using positive ion electrospray ionization with nitrogen as the sheath gas. For tuning, neat solutions of the derivatives were prepared by combining 60 mM CMPI and 5 μg/mL NAC/NACA combined aqueous solution (1:99 by volume). The reaction mixture was infused at 5-10 μL/min into the mobile phase at 0.50 mL/min to identify mass transitions and optimize ionization. Argon was used as the collision gas. For both derivatives, the spray voltage was typically 5000 V, vaporizer temperature, 400° C., and capillary temperature, 250° C.

Thiol stabilization. Sample management for accurate thiol and disulfide measurements in blood or plasma requires prevention of thiol oxidation from the time of venipuncture until analysis. As related guidance, prior publications on assays for endogenous glutathione and its disulfide [7, 8, 11] recommended numerous tactics for stabilization, including acidification of the sample, collection in blood collection tubes with chelating agents, chilling specimen tubes, and masking the thiol to reduce reactivity. For GSH, maintaining intact cellular boundaries is also critical since intracellular concentrations are >100× higher than in the plasma. To prevent artifactual shifts in redox state, Squellerio et al. [12] measured GSH and GSSG in whole blood using K2EDTA collection tubes, immediately acidified the plasma with trichloroacetic acid, and held samples in ice-water temperatures to create a stable injectable supernatant. Carroll et al. [20] quantitated GSH and GSSG directly in mass-limited stem cell lysates by LC-MS/MS; quantitation limits of 1-5 ng/mL were achieved. Limited GSH stability was noted at low concentrations. Moore et al. [21] took protective measures one step further, by reacting GSH with N-ethylmaleimide to irreversibly form a stable thioether; the reagent also contained EDTA and sulfosalicylic acid to deproteinize the sample. GSSG was detected directly.

Consistent with these publications on glutathione, these results with underivatized NAC and NACA thiol in plasma showed that both were unstable when monitored directly using a HILIC LC-MS/MS separation. Half-lives for thiol disappearance were approximately 10 minutes at room temperature. Therefore, the inventors sought to stabilize NAC and NACA by derivatization. When plasma samples spiked with NAC and NACA were treated with TCEP and CMPI, the thiols were well recovered as the thioether derivatives. If TCEP was omitted, recoveries were nil, indicating that the thiols had already been converted to the disulfide forms and unable to react with CMPI.

CMPI was ultimately selected as the derivatization reagent due to its rapid reaction with thiols in aqueous solution. For disulfide reduction, tris(2-carboxyethyl)phosphine (TCEP) was chosen due to its ease of use, unremarkable by-products, and facile reactivity. Dithiothreitol was avoided due to its reactivity with CMPI.

Chromatography. Thiol/disulfide separations have generally been performed using reverse phase (RP) liquid chromatography, ion pair RP HPLC, or HILIC. New and Chan [22] demonstrated that GSSG, ophthalmic acid, and the N-ethylmaleimide GSH derivative were generally well-retained on BEH C18, BEH HILIC and HSS T3 (C18) columns. They recommended these columns be used in isocratic separations for optimal retention time stability and for their analytes preferred the silica-based C18 phase for selectivity and short retention times.

For the method of the present invention and injecting solvent-rich extracts (75% acetonitrile) under RP conditions suitable for retention of these small highly polar derivatives would have caused peak distortion and splitting. To improve selectivity and maintain shorter retention times, a hydrophilic interaction liquid chromatography (HILIC) separation scheme was advantageous, since the solvent-rich extracts could be injected directly (saving time by avoiding a supernatant evaporation step) [23]. Also, the added mass from the derivatization (a single N-methylpyridinyl group) did not appreciably change the relative polarities between the 2 analytes, enabling complete baseline resolution (relative retention factor ˜1.7×). Had a larger reagent tag been employed, such as a polyaromatic maleimide, the selectivity would be less controlled by NAC and NACA and more so by the tags, as shown by Wu et al. [15]. Further testing of the HILIC separation showed only minor ion suppression effects in a survey of 10 different lots of human plasma. These effects were normalized by the isotopic internal standards.

During method development, the inventors noted that spiking solutions were susceptible to degradation in the 0.5 mM disodium EDTA solution, particularly at lower concentrations. For accurate measurement of free thiols, these solutions cannot contain partial amounts of disulfides. Free thiol losses of up to 30% were noted when the spiking solutions were prepared in neutral EDTA solution. Acidification with formic acid (0.1% by volume) stabilized spiking solutions for at least a week at room temperature.

Validation. The method was validated according to regulatory guidances [24-25]. Three independently prepared analytical runs were executed to test the linearity of the calibration curve and the precision and accuracy of measuring validation samples (over 4 concentrations). Additional runs to evaluate recovery, matrix factor/suppression effects, reinjection reproducibility, and carryover were also performed. The selectivity of the method was evaluated over 10 lots of human plasma and the same lots were spiked at the mid QC level to verify inter-subject precision and accuracy. Freeze/thaw, benchtop, and autosampler stabilities were determined. Precision and accuracy data were also generated in lipemic and hemolyzed plasma lots, and stability in whole blood was tested. Long run lengths (up to 192 samples over 2 plates) were evaluated to determine whether the analytical system was reliable over twice the expected duty cycle.

Due to the analytes having two redox states, there were additional experiments conducted to assure that the assay was under control. Available disulfide reference standard was added to control plasma and the samples assayed to verify that the reaction scheme for the total method was working. Untreated and CMPI-treated control plasma were spiked with thiols and incubated at room temperature to permit some oxidation; the samples were then assayed by the total method to verify recovery of the thiol in the untreated samples.

The validation results are summarized in Table 1 for free NAC and NACA and in Table 2 for total NAC and NACA.

TABLE 1
Summary performance data for free NAC/NACA method. All validation
samples (VS) were created from CMPI-fortified human plasma.

| Parameter | NAC (as CMPI derivative) | NACA (as CMPI derivative) |
|---|---|---|
| Validation samples | LLOQ VS (50.0 ng/mL): 0.4% | LLOQ VS (50.0 ng/mL): 0% |
| Inter-run % Bias | Low VS (150 ng/mL): −4% | Low VS (150 ng/mL): -4% |
| | Middle VS (3750 ng/mL): −1.1% | Middle VS (3750 ng/mL): 0.8% |
| | High VS (37,500 ng/mL): −0.5% | High VS (37,500 ng/mL): 2.1% |
| Validation samples | LLOQ VS (50.0 ng/mL): 5.5% | LLOQ VS (50.0 ng/mL): 2.7% |
| Inter-run % CV | Low VS (150 ng/mL): 3.6% | Low VS (150 ng/mL): 2.7% |
| | Middle VS (3750 ng/mL): 3.5% | Middle VS (3750 ng/mL): 2.9% |
| | High VS (37,500 ng/mL): 2.4% | High VS (37,500 ng/mL): 1.8% |
| Dilution Integrity | 100,000 ng/mL diluted 20-fold | 100,000 ng/mL diluted 20-fold |
| | Mean bias: 1%, Precision: 2.4% | Mean bias: 4%, Precision: 1.5% |
| Selectivity | No interferences in 10 lots of matrix, including 2 hemolyzed, and 2 lipemic | |
| Spiked Selectivity | For 10 plasma lots spiked to contain 3750 ng/mL | For 10 plasma lots spiked to contain |
| | Mean bias: −4.0%, Precision: 3.1% | 3750 ng/mL |
| | | Mean bias: −4.3%, Precision: 2.2% |
| Hemolyzed Samples | Acceptable precision and accuracy (less than 4% CV and −3.3% to +0.8% mean bias) | |
| Lipemic Samples | Acceptable precision and accuracy (less than 5% CV and −9.7% to +5.6% mean bias) | |
| Recovery | 59.0-62.1% for NAC and 44.0-53.7% for NAC-D₃ | 89.2-99.2% for NACA and 71.3- |
| | | 89.4% for NACA-D₃ |

| | |
|---|---|
| Benchtop Stability | At least 20 hours at room temperature, either analyte |
| Freeze/Thaw Stability | At least 5 cycles, at either −20° C. or −80° C., either analyte |
| Long-Term Stability | At least 364 days at either −20° C. or −80° C. for NAC; at least 364 days at -80° C. for |
| | NACA. |
| Extract Stability | At least 105 hours for samples maintained at 2-8° C. until injection, either analyte |
| Reinjection Reproducibility | At least 91 hours for samples already injected and then maintained at 2-8° C. |
| Whole Blood Stability | At least 0.96 hours in ice-water, over multiple points |
| Stock Solution Stability | At least 35 days for NAC, and 22 days for NACA in disodium EDTA, when stored at 2- |
| | 8° C., for 5.00 mg/mL concentration. Spiking solutions to be freshly prepared. |

Results for free NAC/NACA method. All calibration standard samples and validation samples were created in CMPI-treated plasma, to match the composition of harvested plasma from study samples. Three precision and accuracy runs were used to determine the best regression fit. Each run included, at a minimum, duplicate calibration standard samples for 8 concentrations, validation samples (4 concentrations), carryover, and negative and positive control samples. The mean bias for any calibration standard was 3.0% or better, and the precision (as % CV) ranged from 0.9 to 3.4%.

TABLE 2
Summary performance data for total NAC/NACA methods. Two types of validation samples (VS, based on either
untreated plasma or plasma pre-treated with CMPI) were tested in the validation runs for the total method.

| Parameter | NAC | NACA |
|---|---|---|
| Validation samples | CMPI pretreated plasma | CMPI pretreated plasma |
| Inter-run % Bias | LLOQ VS (50.0 ng/mL): −2.6% | LLOQ VS (50.0 ng/mL): −0.4% |
| | Low VS (150 ng/mL): −2.7% | Low VS (150 ng/mL): −0.7% |
| | Middle VS (3750 ng/mL): −0.5% | Middle VS (3750 ng/mL): 0.8% |
| | High VS (37,500 ng/mL): −1.9% | High VS (37,500 ng/mL): −0.5% |
| | Untreated plasma | Untreated plasma |
| | LLOQ VS (50.0 ng/mL): −4.6% | LLOQ VS (50.0 ng/mL): −0.4% |
| | Low VS (150 ng/mL): −4% | Low VS (150 ng/mL): −1.3% |
| | Middle VS (3750 ng/mL): −0.5% | Middle VS (3750 ng/mL): −0.5% |
| | High VS (37,500 ng/mL): −2.4% | High VS (37,500 ng/mL): −1.1% |
| Validation samples | CMPI Pretreated | CMPI Pretreated |
| Inter-run % CV | LLOQ VS (50.0 ng/mL): 8.5% | LLOQ VS (50.0 ng/mL): 3.5% |
| | Low VS (150 ng/mL): 4.6% | Low VS (150 ng/mL): 2.6% |
| | Middle VS (3750 ng/mL): 4.1% | Middle VS (3750 ng/mL): 2.1% |
| | High VS (37,500 ng/mL): 3.6% | High VS (37,500 ng/mL): 2.3% |
| | Untreated | Untreated |
| | LLOQ VS (50.0 ng/mL): 8.3% | LLOQ VS (50.0 ng/mL): 4.2% |
| | Low VS (150 ng/mL): 4.8% | Low VS (150 ng/mL): 2.5% |
| | Middle VS (3750 ng/mL): 3.2% | Middle VS (3750 ng/mL): 2.3% |
| | High VS (37,500 ng/mL): 2.5% | High VS (37,500 ng/mL): 2.7% |
| Dilution Integrity | CMPI Pretreated | CMPI Pretreated |
| | Over-range QC: 100,000 ng/mL diluted 20-fold | Over-range QC: 100,000 ng/mL diluted 20- |
| | Mean bias: 0%, Precision: 2.9% | fold |
| | Untreated | Mean bias: 1%, Precision: 1.5% |
| | Over-range QC: 100,000 ng/mL diluted 20-fold | Untreated |
| | Mean bias: 0%, Precision: 3.5% | Over-range QC: 100,000 ng/mL diluted 20- |
| | | fold |
| | | Mean bias: −0.2%, Precision: 1.9% |
| Selectivity | No interferences in 10 lots of matrix, including 2 hemolyzed, and 2 lipemic | |
| Spiked Selectivity | CMPI Pretreated | CMPI Pretreated |
| | For 10 lots spiked to contain 3750 ng/mL | For 10 lots spiked to contain 3750 ng/mL |
| | Mean bias: 1.9%, Precision: 4.5% | Mean bias: −1.3%, Precision: 2.9% |
| | Untreated | Untreated |
| | For 10 lots spiked to contain 3750 ng/mL | For 10 lots spiked to contain 3750 ng/mL |
| | Mean bias: −0.5%, Precision: 3.8% | Mean bias: −4.0%, Precision: 5.3% |
| Hemolyzed Samples | Acceptable precision and accuracy (less than 15% CV and mean bias) | |
| Lipemic Samples | Acceptable precision and accuracy (less than 15% CV and mean bias) | |
| Recovery | 59-71% for NAC and 57-75% for NAC-D₃ | 102-124% for NACA and 102-118% for |
| | | NACA-D₃ |

| | |
|---|---|
| Benchtop Stability | At least 22 hours at room temperature for both CMPI pretreated and untreated |
| Freeze/Thaw | At least 5 cycles, at either −20° C. or −80° C. for both CMPI pretreated and untreated |
| Stability | |
| Long-Term Stability | At least 284 days, at −80° C. for both pretreated and untreated |

To verify accuracy of the total assay, special validation samples were prepared using N, N′-diacetyl-L-cystine at one-half the molar concentration of the middle NAC validation sample (3750 ng/mL). The NAC concentration found was 3800 ng/mL, with a precision of 1.1% CV.

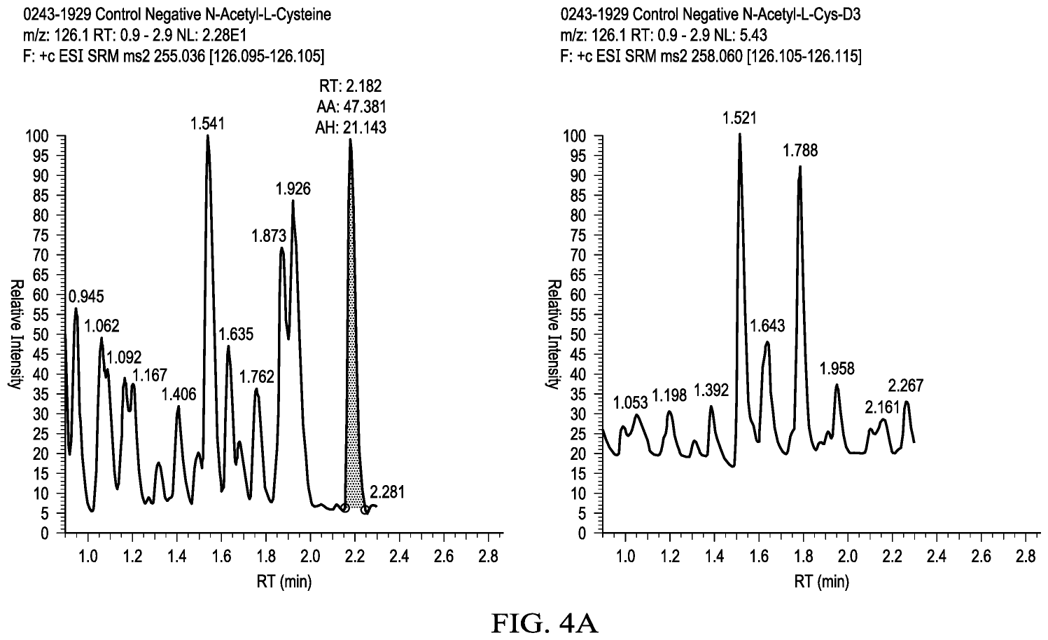

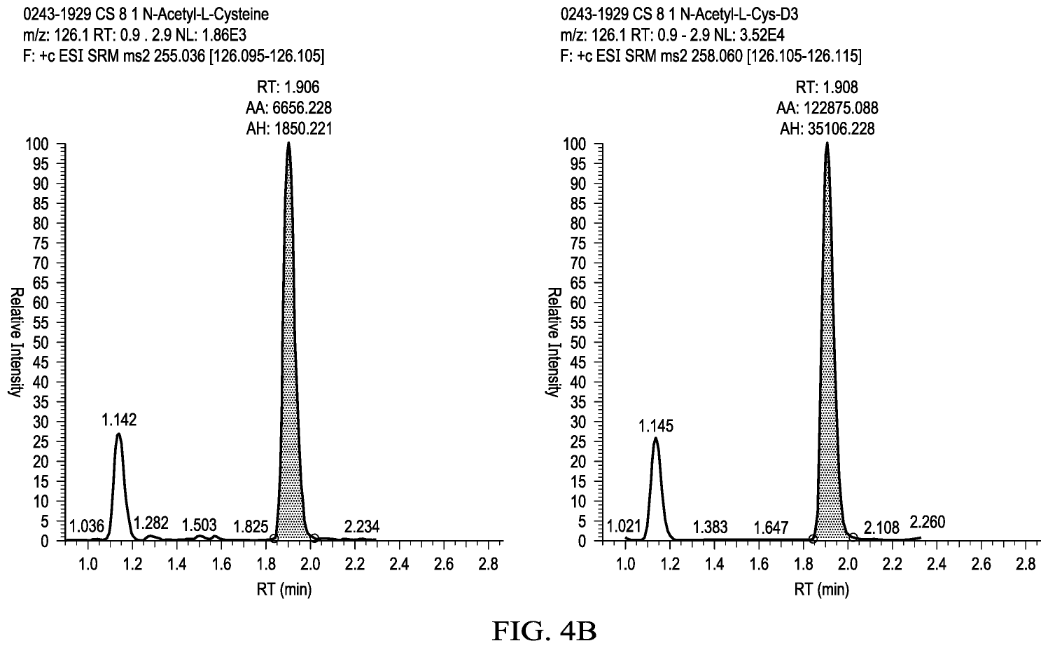

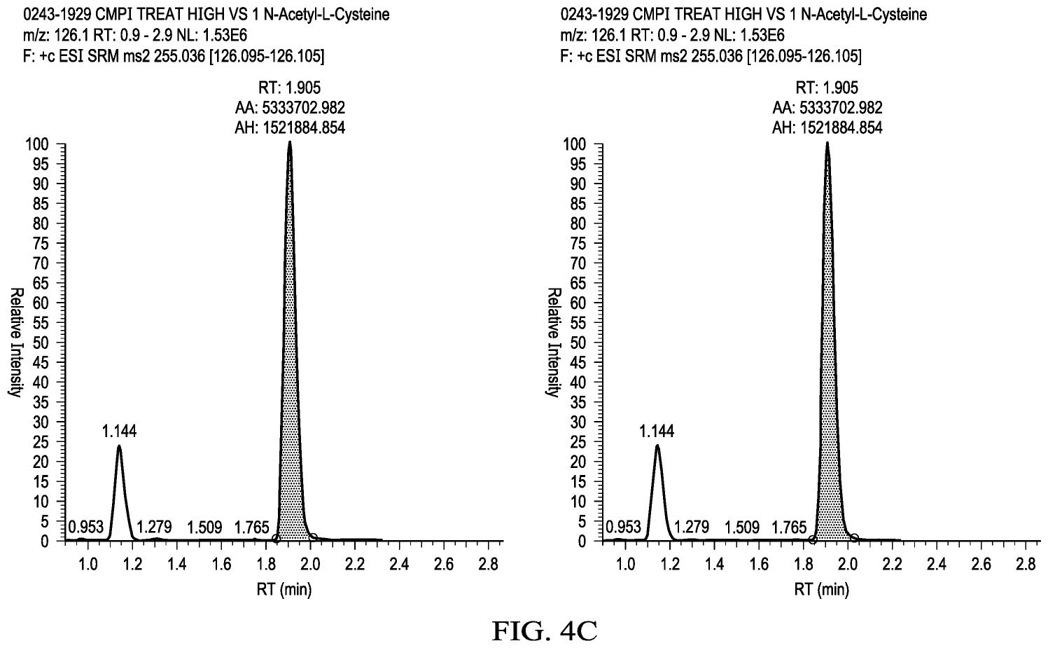

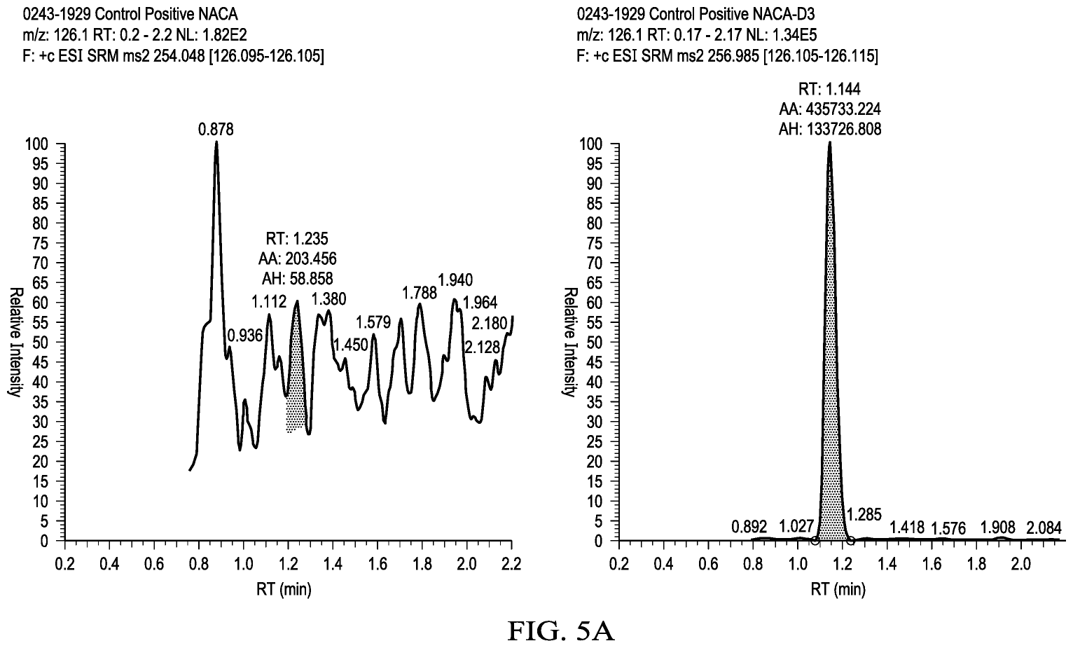

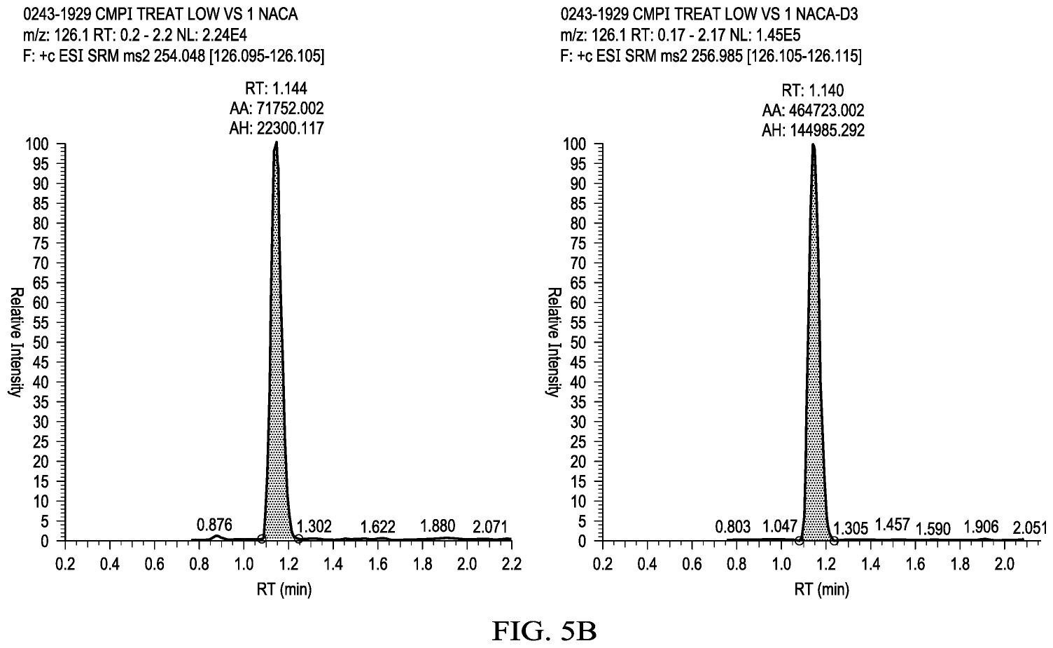

Table 3 shows NAC data; the NACA data was very similar. A linear regression fit was acceptable for both analytes, with correlation coefficients (R2) greater than 0.999. Assorted chromatograms from blank plasma, lower limit calibration standard, and low and high QC samples are shown in FIGS. 4A to 4C and 5A to 5B for NAC and NACA. In FIGS. 4A to 4C, the NACA peak is detected at ˜1.14 minutes under the NAC MS conditions since the masses differ by only 1 unit but the peaks are well separated under HILIC conditions, with the less polar NACA derivative eluting first.

TABLE 3
Calibration standard and regression parameters
for free NAC assay in CMPI- fortified plasma

| | NAC Calibration Standards, Nominal Concentration | | | | | | |
|---|---|---|---|---|---|---|---|
| | 50.0 | 100 | 500 | 2500 | 5000 | 20000 | 50000 |
| | ng/mL | ng/mL | ng/mL | ng/mL | ng/mL | ng/mL | ng/mL |
| Mean Concentration | 50.3 | 99.4 | 491 | 2540 | 4920 | 20400 | 50300 |
| Found | | | | | | | |
| Inter-run % CV | 1.5 | 1.5 | 3.4 | 1.8 | 1.6 | 1.6 | 1.5 |
| Inter-run % Bias | 0.6 | −0.6 | −1.8 | 1.6 | −1.6 | 2 | 0.6 |
| n | 6 | 6 | 6 | 6 | 6 | 6 | 6 |

| Run ID | NAC Slope | NAC Intercept | R-Squared |
|---|---|---|---|
| 13 | 0.00109242 | −0.00112880 | 0.9992 |
| 15 | 0.00112251 | −0.00120828 | 0.9994 |
| 17 | 0.00109341 | −0.00158616 | 0.9995 |

Carryover for both analytes was less than 10% of the lower limit calibration standard. To prove dilution integrity, an over-range validation sample pool (100 μg/mL) was assayed after 20-fold dilution with control plasma, in 6 replicates. The measured concentrations of NAC and NACA after correction for the dilution factor presented mean bias ≤4%, and % CV's ≤2.4%.

To test the selectivity of the method, ten lots of human plasma (from different individuals including 2 hemolyzed and 2 lipemic lots) were evaluated. These lots were fortified with CMPI to simulate plasma harvested from the prescribed blood collection procedure. None of the lots presented significant free NAC or NACA response (less than 1.6% of LLOQ response), to be expected based on likely prior oxidation of any thiol to disulfide. The same lots when spiked to contain 3750 ng/mL NAC and NACA were measured with a mean bias of −4.0 and −4.3% and a precision (% CV) of 3.1 and 2.2%, respectively.

Recoveries were measured by comparing the peak areas for NAC, NACA and their internal standards in validation samples to control plasma extracts spiked to contain amounts of the analytes and internal standards at 100% recovery. For either analyte, the recoveries were consistent across the range of measured concentrations. The recoveries for NAC and its internal standard consistently averaged 50-60%. NACA and its internal standard were recovered 80-95%, presumably due to lower polarity of the amide.

All stability testing in plasma (benchtop, freeze/thaw, and long-term storage) was performed using QC samples prepared in both CMPI-fortified plasma as well as unfortified plasma. In this manner, the stabilities of both the oxidized thiols (in disulfide forms) and the thioether derivatives were tested, since it was assumed that unknown samples would likely contain some of both species. The CMPI-thioethers were stable for at least 5 freeze/thaw cycles and for at least 20 hours at room temperature, as compared to less than 1 hour for the underivatized free thiols. The oxidized thiols were stable for at least 5 freeze/thaw cycles and for at least 22 hours, as evidenced by their full recovery as thioethers when assayed using the total method. Stability was also tested in extracts, whether held for first injection or reinjected. Extracts were stable for over 90 hours when refrigerated. Precision and accuracy was acceptable in lipemic and hemolyzed plasma.

Stability in whole blood was tested to determine whether any delay in the harvesting of plasma would affect the accuracy of the method. Fresh whole blood was fortified with CMPI and then immediately spiked with NAC and NACA to create low and high validation sample pools. These pools were equilibrated at 37° C. for approximately 20 minutes to establish the distribution of analytes between the plasma and erythrocytes. The 2 pools were then held on wet ice and sampled at four times. The first sampling was immediate, and the plasma from each pool was removed and frozen. These samples were designated as the control group. The other samplings occurred at 0.23, 0.49, and 0.96 hours. The plasma samples for each time point were assayed in 3 replicates, all in the same run. Over both analytes, the change from the control group for any sampling interval ranged from +2.4% to −4.7%. There was no meaningful change in analyte concentration for about 1 hour in CMPI-fortified whole blood held on wet ice. The recommended procedure for blood sampling thus entailed: (1) Collection in chilled K2EDTA blood collection tube; (2) Immediate uncapping of the tube and addition of 1.5 M aqueous CMPI (add 1% by volume; for a 3 mL tube add 30 μL); (3) Recapping, gentle mixing, and placement on wet ice for up to 1 hour; and (4) Centrifugation and transfer of the plasma to a storage tube, to be frozen.

Long run lengths were evaluated by preparing two separate 96-well plates of calibration samples and validation samples at the same time and injecting them in sequence (192 samples as a single run). The calibration curve was created from the front curve on the first plate and the back curve at the end of the second plate (16-point regression set). The validation sample concentrations on either plate were computed from this composite curve. All validation sample results met acceptance criteria (Table 4) regardless of location within the long run. The free NAC and total NACA results are shown for illustration of run performance; the accuracy and precision was acceptable throughout the entire run. The total NAC and free NACA results were similarly acceptable.

TABLE 4
Long run length results for free NAC and total NACA assay

| Location in | NAC Validation Samples, | 50.0 | 150 | 3750 | 37500 |
|---|---|---|---|---|---|
| run | Nominal Concentration | ng/mL | ng/mL | ng/mL | ng/mL |
| Plate 1 | Intrarun Mean | 51.2 | 144 | 3820 | 37000 |
| | Intra run % CV | 4.2 | 1.9 | 1.8 | 2.1 |
| | Intrarun % Bias | 2.4 | −4 | 1.9 | −1.3 |
| | n | 6 | 6 | 6 | 6 |
| Plate 2 | Intrarun Mean | 50.1 | 145 | 3820 | 36300 |
| | Intra run % CV | 4.3 | 2.5 | 3.4 | 1.4 |
| | Intrarun % Bias | 0.2 | −3.3 | 1.9 | −3.2 |
| | n | 6 | 6 | 6 | 6 |
| Location in | NACA Validation Samples, | 50.0 | 150 | 3750 | 37500 |
| run | Nominal Concentration | ng/mL | ng/mL | ng/mL | ng/mL |
| Plate 1 | Intrarun Mean | 50.1 | 145 | 3800 | 37400 |
| | Intrarun % CV | 1.8 | 2.4 | 1.1 | 2.2 |
| | Intrarun % Bias | 0.2 | −3.3 | 1.3 | −0.3 |
| | n | 6 | 6 | 6 | 6 |
| Plate 2 | Intrarun Mean | 50.5 | 146 | 3790 | 37300 |
| | Intrarun % CV | 0.6 | 3.2 | 0.6 | 2.4 |
| | Intrarun % Bias | 1.0 | −2.7 | 1.1 | −0.5 |
| | n | 6 | 6 | 6 | 6 |

Validation results for total NAC/NACA method. The “total” method for NAC and NACA converts any disulfide forms of NAC or NACA to the free thiols and then measures the CMPI thioether reaction products. Validation samples were created in CMPI-fortified plasma and unfortified plasma to show that the measurement of total thiol would be accurate regardless of the degree of oxidation prior to analysis. Without CMPI fortification, a significant portion of the thiol spiked into plasma will oxidize to the disulfide form.

Both the free and total assays rely on measurement of the same thioether. For this reason, the extract stability and reinjection reproducibility data reported in section 3.3 apply to both methods. Chromatograms from the total method were identical in appearance to FIGS. 4 and 5. The calibration standard precision and accuracy results using the 1/×2 linear fit were also very similar to the free NAC/NACA data presented in section 3.3.

For untreated matrix across 4 concentrations of validation samples, the bias ranged from −4.6% to −0.4%, and CV's from 2.5% to 8.3%. The validation sample results in treated matrix showed mean bias from −2.7% to +0.8%, with CV's from 2.1% to 8.5%.

N-acetylcysteine is endogenous and is mostly present in the oxidized form. For the free assay, no significant amounts of the reduced thiol were present in a survey of 10 commercial lots of human plasma, even when the lots were fortified with CMPI. In testing the selectivity of the total assay, any endogenous amounts, whether reduced or oxidized, will be converted to the thioether. In a survey of 10 lots of human plasma, the amount of total N-acetylcysteine ranged from 24% to 55% of the LLOQ response. As these lots did not present any measurable free thiol, the endogenous content is from the disulfide form. As NACA is not endogenous, the total NACA response in the same samples was measured at 2.0% or less of LLOQ.

Spiked selectivity was evaluated in both CMPI-fortified and untreated plasma; the lots were spiked with 3750 ng/mL each of NAC and NACA. In the untreated plasma, significant oxidation will be likely at bench temperature, and accurate measurement was only expected with the total assay. The spiked selectivity results were measured with acceptable precision and accuracy in the total assay, regardless of the plasma treatment or analyte (Table 2). The spiked selectivity results were only accurate for CMPI treated samples in the free assay (Table 1).

Thus, a two-stage method was developed to measure free and total N-acetyl-L-cysteine (NAC) and N-acetyl-L-cysteine amide (NACA) in human plasma. Due to facile oxidation of the thiol moiety to the disulfide, a sample collection scheme was devised to protect the thiol by converting it to a stable thioether. Prior methods have typically measured “total thiol” due to the instability of thiols in blood plasma. The proposed method immediately converts reduced thiols to a charged thioether amenable to an efficient HILIC-based separation, which allows the solvent-rich extracts from the protein precipitation to be injected directly, without the expected peak distortion from a reverse phase separation. The derivatives are conveniently made with inexpensive reagents under aqueous conditions and are easy to implement in any laboratory. The method also conserves the sample by processing the plasma for thiol first using CMPI derivatization, and then taking an intermediate portion of the work-up for TCEP reduction of disulfides to free thiols followed by derivatization with CMPI.

Only a single 25 μL sample is required and the entire procedure is executed in 96-well plates. Methods with less sensitive HPLC instruments have required separate samples (as much as 500 μL) to measure each redox state. The same chemistry and separation conditions may be applied to the analysis of endogenous thiols such as cysteine, homocysteine, and glutathione (unpublished data) and thiol-containing drugs such as captopril. Smaller analytes such as cysteine and cystine will be well-retained compared to conventional reverse phase or ion pair separations, and due to the UHPLC format, the overall separation time will only be a few minutes. Tandem mass spectrometry provides equivalent (or better) selectivity than existing HPLC methods with optical or electrochemical detection, thus, that this sample preparation method is directly applicable to analyses with higher mass resolution instruments. The method has been successfully utilized in Phase 1 clinical trials of NAC [26] (112 samples for total NAC) and NACA [27] (1098 plasma samples assayed for total NACA and NAC). In the latter study, incurred sample reanalysis demonstrated repeatabilities of 99.1% for NACA and 95.5% for NAC.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

[1] Drugs@FDA: FDA Approved Drug Products, accessed Sep. 14, 2018 at: https://www.accessdata.fda.gov/scripts/cder/daf/.
[2] T A Martin, D H Causey, A L Scheffner, A G Wheeler, J R Corrigan. Amides of N-acetylcysteines as mucolytic agents. J Med Chem, 10 (1967) 1172-1176.
[3] K Sunitha, M Hemshekhar, R M Thushara, M Sebastin Santhosh, M Yariswamy, K Kemparaju, K S Girish. N-acetylcysteine amide: a derivative to fulfill the promises of N-acetylcysteine. Free Radic Res, 47 (2013) 357-367.
[4] A Dong, R Stevens, S Hackett, P A Campochiaro. Compared with N-acetylcysteine (NAC), N-acetylcysteine amide (NACA) provides increased protection of cone function in a model of retinitis pigmentosa. Investigative Ophthalmology & Visual Science, 55 (2014) 1736.
[5] A M Schimel, L Abraham, D Cox, A Sene, C Kraus, D S Dace, N Ercal, R S Apte. N-acetylcysteine amide (NACA) prevents retinal degeneration by up-regulating reduced glutathione production and reversing lipid peroxidation. Am J Pathol, 178 (2011) 2032-2043.
[6] M Isokawa, T Kanamori, T Funatsu, M Tsunoda. Analytical methods involving separation techniques for determination of low-molecular-weight biothiols in human plasma and blood. J Chrom B, 964 (2014) 103-115.
[7] P Monostori, G Wittmann, E Karg, S Turi. Determination of glutathione and glutathione disulfide in biological samples: an in-depth review. J Chrom B, 877 (2009) 3331-3346.
[8} K. Kusmierek, G. Chwatko, R. Glowacki, P Kubalczyk, E. Bald, Ultraviolet derivatization of low-molecular-mass thiols for high performance liquid chromatography and capillary electrophoresis analysis, J Chrom B, 879 (2011) 1290-1307.
[9] J H Suh, R Kim, B Yavuz, D Lee, A Lal, B Ames, M K Shigenaga, Clinical assay of four thiol amino acid redox couples by L C-M S/M S: utility in thalassemia, J Chrom B, 877 (2009) 3418-3427.
[10] M E McMenamin, J Himmelfarb, T D Nolin, Simultaneous analysis of multiple aminothiols in human plasma by high performance liquid chromatography with fluorescence detection, J Chrom B, 877 (2009) 3274-3281.
[11] D Giustarini, D Tsikas, G Columbo, A Milzani, I Dalle-Donne, P Fanti, R Rossi, Pitfalls in the analysis of the physiological antioxidant glutathione (GSH) and its disulfide (GSSG) in biological samples: an elephant in the room. J Chrom B., 1019 (2016) 21-28.
[12] I Squellerio, D Caruso, B Porro, F Veglia, E Tremoli, V Cavalca. Direct glutathione quantification in human blood by LC-M S/M S: comparison with HPLC with electrochemical detection. J Pharm Biomed Anal, 71 (2012) 111-8.
[13] K Kusmierek, E Bald. Reversed-phase liquid chromatography method for the determination of total plasma thiols after derivatization with 1-benzyl-2-chloropyridinium bromide, Biomedical Chromatography, 23 (2009) 770-775.
[14] N Ercal, S Oztezcan, T C Hammond, R H Matthews, D R Spitz, High-performance liquid chromatography assay for N-acetylcysteine in biological samples following derivatization with N-(1-pyrenyl)maleimide. J Chrom B, 685 (1996) 329-334.
[15] W Wu, G Goldstein, C Adams, R H Matthews, N Ercal. Separation and quantification of N-acetyl-L-cysteine and N-acetyl-cysteine-amide by HPLC with fluorescence detection. Biomed Chromatogr, 20 (2006) 415-422.
[16] M J Nozal, J L Bernal, L Toribio, P Marinero, O Moral, L Manzanas, E Rodriguez, Determination of glutathione, cysteine, and N-acetylcysteine in rabbit eye tissues using high-performance liquid chromatography and post-column derivatization with 5,5′-dithiobis(2-nitrobenzoic acid). J Chrom A, 778 (1997) 347-353.
[17] C Celma, J A Allue, J Prunonosa, C Peraire, R Obach, Determination of N-acetylcysteine in human plasma by liquid chromatography coupled to tandem mass spectrometry. J Chrom A, 870 (2000) 13-22.
[18] M Katz, S Joon W, Y Park, A Orr, D P Jones, R A Swanson, G A Glass. Cerebrospinal fluid concentrations of N-acetylcysteine after oral administration in Parkinson's disease. Parkinsonism and Related Disorders, 21 (2015) 500-503.
[19] R C Reyes, G F Cittolin-Santos, J-E Kim, S J Won, A M Brennan-Minnella, M Katz, G A Glass, R A Swanson. Neuronal glutathione content and antioxidant capacity can be normalized in situ by N-acetyl cysteine concentrations attained in human cerebrospinal fluid, Neurotherapeutics, 13 (2016) 217-225.
[20] D Carroll, D Howard, H Zhu, C M Paumi, M Vore, S Bondada, Y Liang, C Wang, D K St. Clair, Simultaneous quantitation of oxidized and reduced glutathione via LC-MS/MS: An insight into the redox state of hematopoietic stem cells. Free Radical Biology and Medicine, 97 (2016) 85-94.
[21] T Moore, A Le, A-K Niemi, T Kwan, K Cusmano-Ozoga, G M Enns, T M Cowan. A new LC-MS/MS method for the clinical determination of reduced and oxidized glutathione from whole blood. J Chrom B, 929 (2013) 51-55.
[22] L S New and ECY Chan. Evaluation of BEH C18, BEH HILIC, and HSS T3 (C18) Column Chemistries for the UPLC-MS-MS Analysis of Glutathione, Glutathione Disulfide, and Ophthalmic Acid in Mouse Liver and Human Plasma. J Chrom Sci, 46 (2008) 209-214.
[23] Naidong Weng, Bioanalytical liquid chromatography tandem mass spectrometry methods on underivatized silica columns with aqueous/organic mobile phases. J Chrom B, 796 (2003) 209-224.
[24] Bioanalytical Method Validation, Guidance for Industry, US Dept of Health and Human Services, Food and Drug Administration, CDER/CVM, May 2018.
[25] Guideline on Bioanalytical Method Validation, European Medicines Agency, 21 Jul. 2011, EMEA/CHMP/EWP/192217/2009 Rev1. Corr.2.
[26] ClinicalTrials.gov Identifier: NCT03063021, accessed Dec. 13, 2018 at: https://clinicaltrials.gov.
[27] Australian New Zealand Clinical Trial Registry, ACTRN12617000911392, accessed Dec. 5, 2018 at: http://www.anzctr.org.au.

What is claimed is:

1. A method of detecting free and total NAC, NACA, or both in a biological sample comprising:
adding 2-chloro-1-methylpyridinium iodide (CMPI) to a biological sample suspected of having NAC or NACA to convert free thiols into stable thioethers;
precipitating the protein in the sample;
extracting the stable thioethers and separating into a first and a second extract;
detecting the thioether derivatives from the first extract with LC-MS/MS;
reducing from the second extract free thiols by adding tris(2-carboxyethyl)phosphine (TCEP) followed by converting to stable thioethers with CMPI;
detecting the disulfides reduced to free thioether derivatives from the second extract with LC-MS/MS; and
calculating from the LC-MS/MS and TCEP of the first and second extracts a free and a total sample NAC or NACA.

2. The method of claim 1, wherein the biological sample is a plasma, serum, vitreous humor, tear, sputum, urine, or fecal sample.

3. The method of claim 1, wherein the LC-MS/MS is Liquid Chromatography/Triple Quadrupole Mass Spectroscopy.

4. The method of claim 1, wherein the results provide total assay measures a sum of free plus oxidized NAC or NACA.

5. The method of claim 1, further comprising the step of optimizing the thiol and disulfide measurements by acidifying the sample prior to adding CPMI.

6. The method of claim 1, further comprising the step of performing the ionization at a spray voltage of 5000 V, vaporizer temperature of 400° C., and capillary temperature or 250° C.

7. A method comprising:
measuring by Liquid Chromatography/Triple Quadrupole Mass Spectroscopy (LC-MS/MS) the levels of total NAC, NACA, or di-NACA in a biological sample obtained from a human subject having retinitis pigmentosa, age-related macular degeneration, diabetic retinopathy, myopia, high myopia, Fuchs' dystrophy, diabetic macular edema (DME), geographic atrophy, Stargardt's disease, cataracts, or retinal vein occlusion (RVO), by:
adding 2-chloro-1-methylpyridinium iodide (CMPI) to the biological sample suspected of having NAC or NACA to convert free thiols into stable thioethers;
precipitating the protein in the sample;
extracting the stable thioethers and separating into a first and a second extract;
detecting the thioether derivatives from the first extract with LC-MS/MS;
reducing from the second extract free thiols by adding tris(2-carboxyethyl)phosphine (TCEP) followed by converting to stable thioethers with CMPI;
detecting the disulfides reduced to free thioether derivatives from the second extract with LC-MS/MS; and
calculating from the LC-MS/MS and TCEP of the first and second extracts a free and a total sample NAC, NACA. or di-NACA.

8. The method of claim 7, wherein the biological sample comprises a plasma, serum, vitreous humor, tear, sputum, urine, or fecal sample.

9. The method of claim 7, wherein the human subject is determined to be at risk of developing retinitis pigmentosa or a disorder associated with the eye.

10. The method of claim 7, wherein the human subject is determined to be at risk of developing retinitis pigmentosa or a disorder associated with the eye.

11. The method of claim 7, wherein the human subject is determined to be at risk of developing complications from retinitis pigmentosa or a disorder associated with the eye.

12. The method of claim 7, further comprising calculating the risk or rate of the human subject developing retinitis pigmentosa or a disorder associated with the eye, wherein the risk or rate is calculated based on probability and odds ratios of developing biopsy documented retinitis pigmentosa.

13. The method of claim 7, further comprising providing recommended treatment options for the human subject based on the calculated risk or rate of developing retinitis pigmentosa or a disorder associated with the eye.

14. The method of claim 7, further comprising compiling the calculations of the risk or rate of developing retinitis pigmentosa or a disorder associated with the eye in the human subject into a report.

15. The method of claim 14, wherein the report is transmitted to a third party or to the human subject.

16. The method of claim 15, wherein the transmitting of the report is done over a network.

17. The method of claim 14, wherein the report comprises a risk profile.

18. The method of claim 14, wherein the report is transmitted to a third party and to the subject.

19. The method of claim 7, wherein the human subject has not been diagnosed with retinitis pigmentosa or a disorder associated with the eye.

20. The method of claim 7, wherein a third party obtains the plasma sample from the subject.

21. The method of claim 7, wherein the human subject is undergoing treatment with at least one of NAC, NACA, or diNACA.
